(19) United States
(12) Patent Application Publication
(10) Pub. No.: US 2025/0295649 A1
(43) Pub. Date: Sep. 25, 2025

(54) METHOD FOR PROMOTING ELONGATION OF LONG BONE DURING CHILDHOOD TO ADOLESCENCE BY GPER-1 ACTIVATION VARIANT

(71) Applicant: Kaohsiung Medical University, Kaohsiung City (TW)

(72) Inventors: Mei-Ling Ho, Kaohsiung City (TW); Je-Ken Chang, Kaohsiung City (TW); Chung-Hwan Chen, Tainan City (TW); Ya-Shuan Chou, Taipei City (TW); Shu-Chun Chuang, Kaohsiung City (TW)

(73) Assignee: Kaohsiung Medical University, Kaohsiung City, TW (TW)

(21) Appl. No.: 18/613,108

(22) Filed: Mar. 22, 2024

(51) Int. Cl. A61K 31/473; A61K 31/138; A61K 31/4535; A61K 31/566; A61K 45/06; A61P 19/00

(52) U.S. Cl. CPC A61K 31/473; A61K 31/138; A61K 31/4535; A61K 31/566; A61K 45/06; A61P 19/00

(57) ABSTRACT

A method is proposed to increase the proliferation of growth plate chondrocytes in children from early childhood to adolescence or in subjects where the growth plates are not closed by administering an agonist to activate G protein-coupled estrogen receptor 1 (GPER-1), thereby promoting children to grow taller or enhancing bone growth.

### FIELD OF THE INVENTION

The present invention relates to the application of molecular biology and pharmacology, particularly a method for promoting growth plate chondrocyte proliferation in children through the activation of GPER-1.

### BACKGROUND OF THE INVENTION

The growth plate located at the upper and lower ends of the long bones, also known as the epiphyseal plate, is the key factor influencing individual height and also regulates for human bone growth. Cell proliferation in the growth plate is associated with the elongation of the long bones. The chondrocytes of the epiphyseal plate continue to proliferate and differentiate, leading to a continuous increase in bone length. During human development, the proliferation and ossification of chondrocytes will continue until early adulthood. When girls experience menstruation or boys start to undergo voice changes, it indicates the onset of puberty. The emergence of secondary sexual characteristics indicates the transition to late puberty and the conclusion of the height growth period. The growth plate will be replaced by bone tissue and then closed, causing the backbone to stop “growing” and, consequently, cease increasing in height.

The regulation of the entire bone growth process involves numerous physiological reactions. In addition to exercise and dietary habits, the influence of the endocrine system also plays a significant role. Among of them, sex hormones have an important impact on increased height during adolescence. In the early stages of development, the secretion of trace amounts of estrogen and androgens causes bones to begin to grow rapidly, which is also one of the reasons for the development of sexual characteristics during puberty. As puberty continues to progress, the concentration of estrogen in the body becomes higher and higher, and high concentrations of estrogen pass through the estrogen receptor alpha causing the growth plates to close, ending the growth phase of the child's height. Therefore, a new method is needed to promote the proliferation of chondrocytes effectively and specifically in the growth plate of adolescent children.

Increasing height is a focus that many people pay attention to during the development of children. However, there are still areas for intervention in the regulation of height growth, starting from the physiological mechanism. Current methods of regulating growth plate proliferation to promote height growth include growth hormone treatment and sex-releasing hormone analogue (GnRH analogue) treatment. Growth hormone therapy promotes the elongation of bone growth plates by administering synthetic growth hormone, while sex hormone analogs achieve the same goal by inhibiting the process of individual puberty, but these methods also have a great impact on the physiological response of the whole body influence, such as prone to side effects. Therefore, there is still a need for more effective, safe, and economical methods to promote growth plate chondrocytes proliferation in adolescent children and provide a more specific effect on individual bone growth.

### BRIEF DESCRIPTION OF THE DRAWINGS

FIG. 1 shows the analysis of tibial growth rates in mice from 4 to 8 weeks old after subcutaneous injection of different concentrations of GPER-1 agonists at 2 weeks old to activate their GPER-1. The control group is blank, consisting of mice injected with physiological saline.

FIG. 2 shows the analysis of tibial length in mice at 6 weeks old after subcutaneous injection of 10⁻⁴ g/kg GPER-1 agonist at 2 weeks old, activating their GPER-1. The control group is blank, consisting of mice injected with physiological saline.

FIG. 3A shows the stained images of cartilage tissues in mice at 2 weeks old after subcutaneous injection of different concentrations of GPER-1 agonists, stained with Safranin O. FIG. 3B shows the composition analysis of cartilage tissues in mice at 2 weeks old after subcutaneous injection of different concentrations of GPER-1 agonists. Abbreviations: RZ for the resting zone, PZ for the proliferation zone, HZ for the hypertrophy zone. The control group is blank, consisting of mice injected with physiological saline.

FIG. 4 shows the analysis of the proportion of proliferating cells in the ex vivo cultured epiphyseal cartilage of rat treated with different concentrations of GPER-1 agonists to activate GPER-1, as detected by Bromodeoxyuridine (5-bromo-2′-deoxyuridine, BrdU) labeling.

### DETAILED DESCRIPTION OF THE INVENTION

Other features and advantages of the invention will be revealed from the following non-limiting detailed description and examples.

In order that the present invention may be more readily understood, certain terms are first defined. Additional definitions are set forth throughout the detailed description.

G-protein-coupled estrogen receptor 1 (GPER-1, GPR30) is an estrogen receptor identified in 2005, expressed in the growth plates of both boys and girls. The research indicates that its physiological effects during adolescence impact both boys and girls. The research indicates that GPER-1 primarily influences the growth plates chondrocytes proliferation during adolescence. Due to the time-limited nature of bone growth, it is crucial to choose an appropriate intervention time. Therefore, during the childhood to adolescence period, approximately 6-12 years old, which is also before the closure of growth plates, administering compounds or agonists that activate GPER-1 can stimulate the proliferation of chondrocytes in the growth plates and increase the thickness of the growth plates, thereby promoting height growth in children.

In addition to being physiologically regulated by estrogen, GPER-1 can be targeted individually by drugs or agonists that activate GPER-1. When regulating GPER-1, these substances do not affect other estrogen receptors, thereby reducing side effects on other physiological responses.

As used herein, “a,” “an,” “the,” “at least one,” and “one or more” are used interchangeably.

The present invention provides a method for increasing the thickness of a growth plate in a subject, comprising administering to the subject an effective amount of a GPER-1 agonist, wherein the subject is one in whom the growth plates are not closed or are underdeveloped.

In one embodiment, the subject's age is between 6 and 16 years old. In a preferred embodiment, the subject's age is between 8 and 14 years old. In a more preferred embodiment, the subject's age is between 10 and 12 years old.

In one embodiment, increasing the thickness of a growth plate results from the activation of GPER-1, promoting chondrocyte proliferation in the growth plates.

In another embodiment, increasing the thickness of a growth plate promotes the subject's height growth.

In one embodiment, the GPER-1 agonists include (±)-1-[(3aR*,4S*,9bS*)-4-(6-Bromo-1,3-benzodioxol-5-yl)-3a,4,5,9b-tetrahydro-3H-cyclopenta[c]quinolin-8-yl]-ethenone, estrogen, Tamoxifen, Raloxifene or selective estrogen receptor modulators (SERMs). In a preferred embodiment, the GPER-1 agonist is ((±)-1-[(3aR*,4S*,9bS*)-4-(6-Bromo-1,3-benzodioxol-5-yl)-3a,4,5,9b-tetrahydr 0-3H-cyclopenta[c]quinolin-8-yl]-ethenone.

In another embodiment, the administration of the GPER-1 agonist includes subcutaneous injection, oral administration, or intravenous injection.

In another embodiment, the effective amount of the GPER-1 agonist is 10⁻⁸ g/kg to 10⁻² g/kg. In a preferred embodiment, the effective amount of the GPER-1 agonist is 10⁻⁶ g/kg to 10⁻⁴ g/kg. In a more preferred embodiment, the effective amount of the GPER-1 agonist is 10⁻⁴ g/kg.

### EXAMPLE

The following embodiments are non-limiting and only represent various aspects and features of the present invention.

### Embodiment 1: The Impact of GPER-1 on Tibia Growth Rate

From 2 weeks old, mice are administered GPER-1 agonists at concentrations of 10⁻⁸ g/kg, 10⁻⁶ g/kg, and 10⁻⁴ g/kg through subcutaneous injection to activate GPER-1. The tibia length of mice is analyzed using computed tomography to examine the changes in tibia length of mice between 4 to 8 weeks old.

The GPER-1 agonist is (±)-1-[(3aR*,4S*,9bS*)-4-(6-Bromo-1,3-benzodioxol-5-yl)-3a,4,5,9b-tetrahydro-3H-cyclopenta[c]quinolin-8-yl]-ethenone (GPR30 agonist, G-1).

The tibia growth rate (Linear growth rate) of mice from 4 to 8 weeks old=(8-week-old tibia length (L_{8W})-4-week-old tibia length (L_{4W}))/L_{4W}.

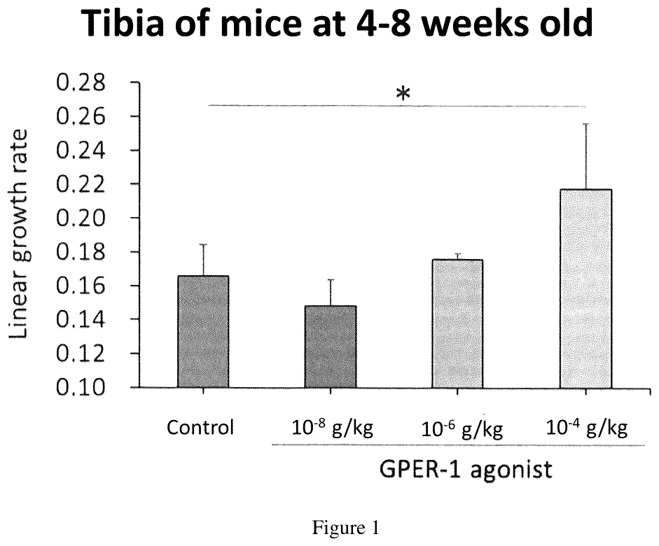

The results, as shown in FIG. 1, indicate a significant enhancement in the tibial growth rate of mice aged 4-8 weeks with the GPER-1 agonist at a dosage of 104 g/kg.

### Embodiment 2: The Impact of GPER-1 on Tibia Length

From 2 weeks old, mice are administered GPER-1 agonists at concentrations of 104 g/kg to activate GPER-1. The tibia length of mice is analyzed using computer tomography at 6 weeks old.

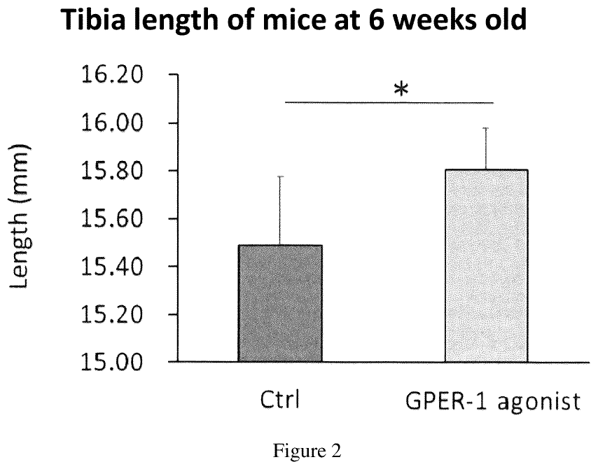

The results, as shown in FIG. 2, demonstrate that GPER-1 agonists can significantly promote the longitudinal growth of the tibia.

### Embodiment 3: Effect of GPER-1 on Growth Plate Cartilage Structure

From 2 weeks old, mice are administered GPER-1 agonists at concentrations of 10⁻⁸ g/kg, 10⁻⁶ g/kg, and 10⁻⁴ g/kg through subcutaneous injection to activate GPER-1. Safranin O staining is employed to label cartilage tissue for the analysis of growth plate cartilage structure. The growth plate cartilage is categorized into the resting zone (RZ), proliferation zone (PZ), and hypertrophy zone (HZ).

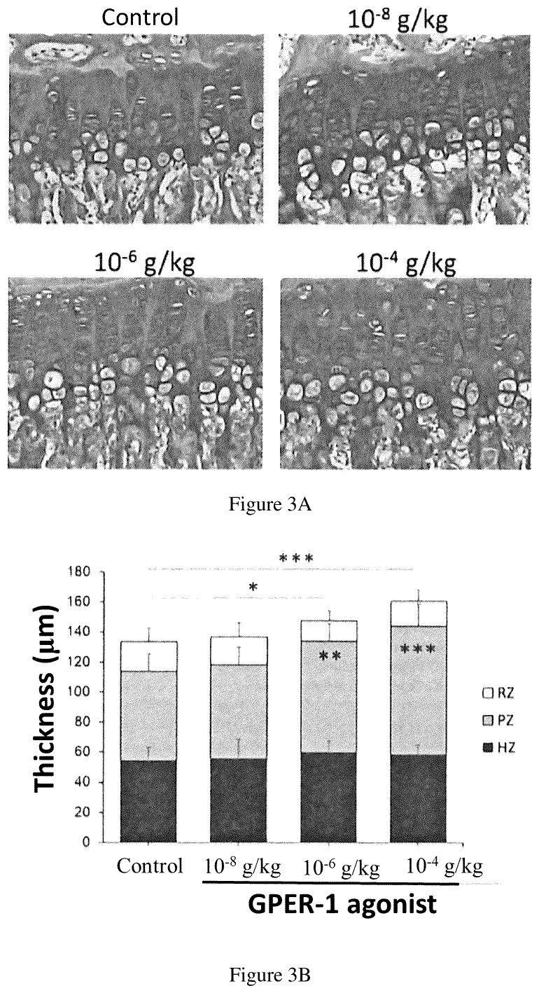

The results, as shown in FIGS. 3A and 3B, indicate that GPER-1 agonists at concentrations of 10⁻⁶ g/kg and 10⁻⁴ g/kg effectively promote the thickness of the proliferative zone of the growth plate and whole growth plate. This suggests that chondrocytes in the growth plate are influenced by GPER-1 agonists at concentrations of 10⁻⁶ g/kg and 10⁻⁴ g/kg, thereby promoting chondrocyte proliferation.

### Embodiment 4: The Impact of GPER-1 on Chondrocytes

After taking out the cultured epiphyseal cartilage from 4-day-old rats and culturing them ex vivo, the cartilage tissues are given GPER-1 agonists at concentrations of 0.1, 0.5, 1, and 2 μM to activate the GPER-1 of chondrocytes. Subsequently, the amount of proliferating cells in living cells is detected using Bromodeoxyuridine (5-bromo-2′-deoxyuridine, BrdU).

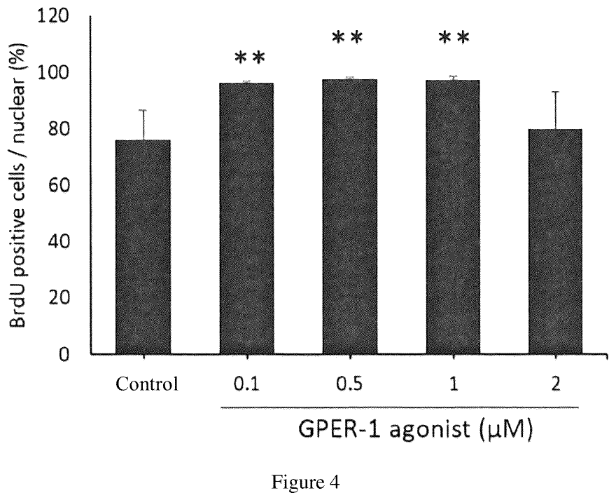

The results, as shown in FIG. 4, indicate that GPER-1 agonists at concentrations of 0.1 and 0.5 μM significantly promote the proliferation of epiphyseal chondrocytes.

The activation of GPER-1 by subcutaneous injection of a GPER-1 agonist is utilized to enhance bone growth. When the dose of the GPER-1 agonist is at 10⁻⁴ g/kg, it stimulates the tibial growth rate and bone length in mice from 4 to 8 weeks old. Additionally, doses of the GPER-1 agonist at 10⁻⁶ g/kg and 10⁻⁴ g/kg promote the thickness of the growth plate and the increase of the proliferative layer in mice. The epiphyseal cartilage tissues from 4-day-old rats are extracted for ex vivo cultivation and administered different concentrations of GPER-1 agonist to activate GPER-1, demonstrating that GPER-1 agonists at concentrations ranging from 0.1 to 1 μM can promote the proliferation of rat chondrocytes.

Although the present invention has been described and illustrated in sufficient detail for those skilled in the art to make and use it, various alternatives, modifications, and improvements should be apparent without departing from the spirit and scope of the present invention.

Those skilled in the art will readily appreciate that the present invention is well suited to achieve the above objects and obtain the above-mentioned advantages, as well as those inherent therein. The processes and methods for their production described above represent preferred embodiments are illustrative and do not limit the scope of the invention. Persons skilled in the art will contemplate modifications and other uses thereof. Such modifications are within the spirit of the present invention and are encompassed by the scope of the claims.

What is claimed is:

1. A method for increasing the thickness of a growth plate in a subject, comprising administering to the subject an effective amount of a GPER-1 agonist, wherein the subject is one in whom the growth plates are not closed or are underdeveloped.

2. The method of claim 1, wherein increasing the thickness of a growth plate results from the activation of GPER-1, promoting chondrocyte proliferation in the growth plates.

3. The method of claim 1, wherein the GPER-1 agonists include (±)-1-[(3aR*,4S*,9bS*)-4-(6-Bromo-1,3-benzodioxol-5-yl)-3a,4,5,9b-tetrahydro-3H-cyclopenta[c]quinolin-8-yl]-ethenone, estrogen, Tamoxifen, Raloxifene or selective estrogen receptor modulators (SERMs).

4. The method of claim 1, wherein increasing the thickness of a growth plate promotes the subject's height growth.

5. The method of claim 1, wherein the administration of the GPER-1 agonist includes subcutaneous injection, oral administration, or intravenous injection.
